(19) United States
(12) Patent Application Publication
(10) Pub. No.: US 2002/0177614 A1
(43) Pub. Date: Nov. 28, 2002

(54) Methods for treating nuerodegenerative diseases including alzheimer's

(76) Inventors: Carl R. Merril, Bethesda, MD (US); Hossein A. Ghanbari, Potomac, MD (US)

Correspondence Address: Mary E. Gormley, Shaw Pittman LLP, 1255 23rd Street NW, Washington, DC, 20037-1128, US

(21) Appl. No.: 09/816,289

(22) Filed: Mar. 23, 2001

(51) Int. Cl. A61K 39/395; A61K 39/38; A61K 31/445

(52) U.S. Cl. 514/355; 424/141.1; 424/184.1

(57) ABSTRACT

Methods of preventing neural tissue damage caused by ADP-ribosylation of eucaryotic elongation factor-2 (EF-2) are disclosed. These methods are especially useful in the treatment of neurodegenerative diseases, such as Alzheimer's disease, Parkinson's disease, and other cognitive disorders of mammals. Also described are methods to screen for drugs that would be useful for treating such diseases.

### FIELD OF THE INVENTION

The present invention is directed to methods of preventing neural tissue damage caused by ADP-ribosylation of eucaryotic elongation factor-2 (EF-2). These methods are especially useful in the treatment of neurodegenerative diseases, such as Alzheimer's disease, Parkinson's disease, and other cognitive disorders of mammals.

### BACKGROUND OF THE INVENTION

Neurodegenerative diseases have a major impact on society. For example, approximately 4 million Americans and 12 million individuals worldwide are afflicted with a chronic neurodegenerative disease known as Alzheimer's disease (AD). Other examples of chronic neurodegenerative diseases include motor neuron disease, Lewy body dementia, diabetic peripheral neuropathy, multiple sclerosis, amyotrophic lateral sclerosis, Huntington's disease, Parkinson's disease, and probably schizophrenia and other neurodegenerative dementias. There is a need for improved therapeutic agents and methods for reversing or retarding neuronal damage associated with each of these conditions.

Most of the neurodegenerative diseases noted above are associated with aging. For example, Alzheimer's disease, which is characterized by symptoms such as depression, aggression, impairment in short-term memory, impairment in intellectual ability, agitation, irritability and restlessness, generally presents itself in the fifth or sixth decade of life. The onset Parkinson's disease is typically after age 40, with increasing incidence with age. Similarly, motor neuron diseases such as ALS occur after age 40.

A common feature of neurodegenerative disorders and the process of aging in animals is the progressive cell damage of neurons within the central nervous system (CNS) leading to loss of neuronal activity and cell death (i.e. apoptosis). This loss of activity has been correlated with adverse behavioral symptoms including memory loss and cognitive deficits. At present, the pharmacological therapy of neurodegenerative disorders is limited to symptomatic treatments that do not alter the course of the underlying disease. Moreover, many of the therapeutic agents that have been developed to retard loss of neuronal activity either have toxic side effects or are prevented from reaching their target site because of their inability to cross the blood-brain barrier.

More recently, much of the research in the field of AD has been focused on the amyloid-β peptide, which makes up the plaques that are seen as the pathological hallmark of the disease. The theory that amyloid-β is responsible for AD is based primarily on research showing an association between the peptide and degenerating neurons in the AD brain, and research demonstrating a linkage between mutations in the β-amyloid precursor protein (APP) and some inherited forms of AD, and that these mutations also result in increased synthesis of amyloid-β. See, Mattson et al., Nature, 382:674-675 (1996). More recently, experiments in PDAPP transgenic mice, which overexpress mutant APP, showed that immunization with amyloid-β₄₂ (containing amino acids 1-42 of the peptide) resulted in preventing the development of amyloid plaque formation neuritic dystrophy, and astrogliosis. Schenk et al., Nature, 400:173-177 (1999).

However, while amyloid may be a hallmark of AD, definitive evidence that it is the actual cause of AD in humans is lacking. It is still considered by some in the scientific community that amyloid plaques are one product in a pathway of a more fundamental cellular process.

Meanwhile, because of the current dissatisfaction with the currently available treatments for the above-described neurodegenerative diseases within the affected population, the need continues for a safer, more effective treatment that addresses the underlying etiology and which will either slow the process of neurodegeneration associated with the disease state or even prevent such neurodegeneration altogether.

The present invention provides such an alternative approach to treating neurodegenerative diseases.

### SUMMARY OF THE INVENTION

The present invention is directed to methods of treating neurodegenerative diseases by interfering with toxins that may result in neuronal loss. Such toxins include those that result from infections in the patient by bacteria such as Corynebacterium diphtheria or Pseudomonas aeruginosa.

Specifically, the present methods interfere with the toxic effects of diphtheria toxin (C. diphtheria) or exotoxin A (P. aeruginosa). The effects of these toxins over time can result in neuronal cell death as a result of the ADP-ribosylation of elongation factor-2.

More specifically, the methods of the present invention use chemotherapeutic agents that interfere or reverse the ADP-ribosylation of EF-2, or use immunotherapeutic methods to neutralize the toxins.

In addition, the present invention provides methods for determining whether a particular therapeutic agent is effective in preventing neuron cell death and, thus, useful for treating neurodegenerative disorders.

### DETAILED DESCRIPTION OF THE INVENTION

The present invention is directed to methods for treating a neurodegenerative disease in a mammal, comprising administering to the mammal an agent that will inhibit and/or reverse ADP-ribosylation of elongation factor-2 (EF-2) in the neurons thereof, whereby neuronal degeneration is ameliorated or prevented. The concept of the present invention that neurodegenerative diseases, particularly Alzheimer's disease, are at least in part due to ADP-ribosylation of the protein EF-2 is based on several observations.

Elongation factor-2 mediates the translocation step in peptide-chain elongation by promoting transfer of peptidyl-tRNA from the A- to the P-site of the ribosome and thereby moving the mRNA relative to the ribosome, bringing the next codon into alignment with the A-site. As such, it is an essential component to protein synthesis in the cell, and inactivation of it leads ultimately to cell death.

In Johnson et al., Molecular Brain Research, 15:319-326 (1992), EF-2 obtained from regions of Alzheimer's Disease (AD)—affected brain was shown to have migrated differently than control brain EF-2 on two-dimensional gels. In this paper, the migration difference was interpreted to be due to increased phosphorylation of EF-2 in the diseased brain. However, the Johnson paper does not provide conclusive evidence that ADP-ribosylated EF-2 was associated with the altered migration and that such modification was not present in the brain samples. In a prior paper by Langstrom et al. (Molecular Brain Research, 5:259-269 (1989)), EF-2 from brains of AD patients was shown to have a decreased function in protein synthesis.

Eucaryotic elongation factor-2 normally has a modified amino acid known as a dipthamide (of which the amino acid histidine is the precursor), which is a target of such bacterial toxins as diphtheria toxin (DT) and exotoxin A (ETA) of P. aeruginosa. Both of these toxins have enzymatic activity capable of ADP-ribosylating the dipthamide residue of EF-2, specifically. This ribosylation results in the loss of function of EF-2 and, ultimately, cell death.

Corynebacterium diphtheria (when infected by the beta bacteriophage) is the organism that produces diphtheria toxin, which is an exotoxin that acts as an NAD-dependent ADP-ribosyltransferase specifically catalyzing the ADP-ribosylation of dipthamide on EF-2. Pseudomonas aeruginosa produces ETA, which has the same enzyme activity as DT. See “ADP-Ribosylating Toxins and G Proteins: Insights Into Signal Transduction”, Moss et al., Eds., American Society of Microbiology (1990), which is incorporated herein in its entirety by reference.

Alzheimer's Disease is one that occurs with increased frequency with aging. It is well known that immune functions decline with aging. See Hodes, R. J., Immunol. Reviews, 160:5-8 (1997). Even immunizations received as children or young adults may not be as effective as people age, possibly due to T cell replicative senescence and memory cell maintenance. Pawelec, G. et al., Frontiers in Bioscience, 3:d59-99 (1998). Childhood immunizations for diphtheria, composed of diphtheria toxoid, are standard in many parts of the world The vaccine lasts between five and 10 years, at which time booster vaccination is required. However, it is likely that the middle-aged to elderly do not receive boosters for diphtheria; moreover, even if boosters are obtained, the ability of the immune system to respond adequately to the diphtheria toxin likely diminishes with age.

There is a chronic carrier state for C. diphtheria, which reside in the nose and throat possibly as nonpathogenic organisms or weakened pathogenic organisms whose toxin is kept in check by the immune system, until an age- or immune-disease-related decline. It is interesting that the one subgroup of the population that tends to keep up their immunizations for diphtheria is the military, and the military has a lower rate of age-related cognitive decline. McLay, Robert N., Military Medicine, 165:622-625 (2000). This is suggestive of a role of toxin-producing diphtheria in the pathogenesis of neurodegenerative diseases, such as Alzheimer's disease.

The present inventors have therefore discovered that a likely culprit of neurodegenerative diseases, such as Alzheimer's, is diphtheria toxin or other ADP-ribosylating toxins that are secreted by pre-existing infections with organisms such as C. diphtheria when the body's immune system is no longer capable of effectively neutralizing such toxins. Alternatively, a pre-existing infection of P. aeruginosa, which is also known to exist in a carrier state and carries a very similar toxin, could be the cause of degeneration of nerve cells, and is contemplated as an embodiment of the present invention. Since it is already known that diphtheria toxin causes pathological lesions preferentially in neuronal tissue (as well as the respiratory system, heart and kidneys), and it is not known at this time that ETA has the same preference, the preferred embodiments of the present invention are directed to diphtheria toxin. However, Pseudomonas is known to cause chronic sinusitis and ear infections; this proximity to the brain tissue certainly does not rule out the ETA from the scope of the present invention.

Basis for the concept that the effects of diphtheria toxin can be blocked is found in U.S. Pat. No. 4,882,146, which is incorporated herein by reference. In this patent it is shown that nicotinamide could prevent or reverse the effects of several bacterial ADP-ribosylating toxins, including Diphtheria and Pseudomonas toxins. However, the ′146 patent was directed to using nicotinamide as a therapeutic in acute illness (e.g. diphtheria) to block the toxic effects so that the host has time to build up its own immune defense. That is, it did not recognize that the effects of toxins could be of a chronic, low level infection that could not be adequately met by the host's immune system.

Although not precluded as an adjunctive therapy in the present invention, mere antibiotic therapy would not be preferred as an effective treatment in these circumstances. Antibiotics do not affect the toxin per se, so they would have no effect on the inhibition of EF-2 caused by the toxin. Moreover, in recent years P. aeruginosa has developed antibiotic resistance, which is responsible for the persistent upper respiratory tract infections. The present invention, therefore, is directly primarily to the neutralization of the toxin and/or the interruption or reversal of ADP-ribosylation of EF-2.

Preferably, the method of treatment of the present invention comprises administering an agent that will interfere (i.e. block or reverse) with ADP-ribosylation of EF-2 in a mammal suffering from a neurodegenerative disease. Such agents include reversible and irreversible inhibitors of ADP-ribosylating toxins, including nicotinamide (or its precursor, nicotinic acid) and its analogues (for example, those disclosed in U.S. Pat. No. 4,882,146) Antitoxin preparations against DT and/or ETA would also be effective. In addition, immunization, or booster immunization, of the affected mammal with a diphtheria toxoid preparation is contemplated as an effective way to interfere with the ADP-ribosylating effect of bacterial toxins.

Neurodegenerative diseases include, for example, Alzheimer's disease, Parkinson's disease, schizophrenia, and other degenerative diseases of the nervous system that may involve neuron cell death. The present invention is not limited to human disease states. For instance, dogs are known to suffer from age-associated cognitive disorders similar to Alzheimer's, and would likely benefit from treatment as well. Preferably, the disease to be treated is Alzheimer's disease.

Pharmaceutical treatment is effected by administering to the mammal an anti-ADP-ribosylation amount of a compound, or compounds, which will reverse or block the enzymatic reaction. Methods for determining whether a compound has an inhibitory or reversal affect are known in the art, for instance U.S. Pat. No. 4,882,146, supra, and Iglewski et al., “ADP ribosylation of elongation factor 2 in animal cells”, In ADP-Ribosylating Toxins and G Proteins: Insights Into Signal Transduction, Moss et al., Eds., supra, both of which have been incorporated by reference. Preferred compounds are nicotinamide (or its precursor, nicotinic acid) and its analogues, alone or in combination with methionine or other beneficial compounds, as well as in suitable pharmaceutical carriers. Such pharmaceutical carriers are well known in the art, and formulations depend on factors such as the mode of administration. See Remington's Pharmaceutical Sciences, 18th Ed., Mack Publishing Co. (1990), which is incorporated in its entirety herein by reference. Nasal administration is the preferred route for the treatment of Alzheimer's disease and Parkinson's disease, because it will allow the agent to get directly to the brain. Nasal administration can be in the form of a liquid spray or a powder spray, a gel, ointment, infusion, injection, or nose drops. Liquid or powder sprays are preferred. The agent is inhaled through the nasal passages and absorbed by the nasal mucosa, where in turn the agent will travel through the olfactory neural pathway.

Preferred in the present invention is an immunotherapeutic approach, which involves passive immunotherapy with an antitoxin or vaccination with a toxoid preparation. By “antitoxin” is meant an anti-toxin antibody preparation, and can include polyclonal antiserum raised against the DT and/or ETA, or a preparation containing one or more monoclonal antibodies or fragments thereof which will specifically bind to, and neutralize, the DT and/or ETA. Such antibodies are well known in the art; for example, U.S. Pat. No. 4,689,299, incorporated herein by reference, discloses human monoclonal antibodies against ETA and passive antibody therapy using the same. U.S. Pat. No. 4,689,299, which is incorporated herein by reference, discloses human monoclonal antibodies against diphtheria toxin, which can be used for passive immunotherapy according to the present invention. Particularly preferred in the present invention are increased affinity (as compared to wildtype counterparts) antibodies, more preferably single chain Fv antibodies, which may be produced by the yeast surface display and affinity maturation methods disclosed in WO99/36569A1, which is hereby incorporated by reference.

The term “toxoid” is meant to include a toxin that has been treated to destroy its toxic property without affecting its antigenic properties. Typically, toxins are formalinized to prepare a toxoid. However, in recent years diphtheria toxoids have been prepared by recombinant genetic means, such as disclosed in U.S. Pat. Nos. 4,950,740; 5,843,711; and 5,843,462, each of which is incorporated herein by reference, and all such toxoids and recombinant vaccines are contemplated by the present invention. Toxoids prepared from ETA of P. aeruginosa are disclosed in U.S. Pat. Nos. 4,470,924 and 4,488,991, which are incorporated herein by reference; a chimeric toxoid is disclosed in U.S. Pat. No. 4,771,127, incorporated herein by reference.

Both forms of immunotherapy have been practiced in the art for years. See The Merck Manual, Fifteenth Edition, Berkow et al., Eds. (1987), which is incorporated herein by reference, particularly Chapts. 182 and 191. Indeed, commercial vaccine and antitoxin products already exist, which are useful for the present invention. Diphtheria antitoxin may be administered according to the present invention intravenously or intramuscularly (with horse antitoxin) or intramuscularly (with pooled human gamma-globulin) in a dosage of between 10,000 to 100,000 u. The amount given is decided empirically, depending on the severity of neural degeneration, i.e. disease state. The length of treatment may be a day, a week, or longer and may, as in the case of a chronic progressive illness, last over the entire lifetime of the patient.

Passive immunotherapy provides temporary protection against the toxin being neutralized. Horse globulins containing anti-diphtheria toxin have been extensively employed in humans prophylactically and as treatment, but its use is somewhat restricted in recent years because of the complication of serum sickness developed in response to the horse antibodies. Human gamma-globulin preparations against DT and ETA have also been employed, which are derived from the serum of individuals that have been vaccinated a few weeks previously. Care must also be taken, however, with human antisera as it can cause anaphylactic reactions when administered intravenously; therefore, intramuscular injection is required.

Preferably, an anti-DT and/or anti-ETA monoclonal antibody preparation is employed for passive immunotherapy. More preferably, the monoclonal antibodies are human. Such human monoclonal antibodies are known in the art, as disclosed in, for example, U.S. Pat. No. 4,689,299, supra. The monoclonal antibodies may be suspended or dissolved in any suitable liquid carrier, e.g., water or a saline solution, and administered parenterally, orally, intraocularly or intranasally. Preferably, the monoclonal antibodies are administered intranasally for the same reason given above with respect to the chemotherapeutic agents (i.e. delivery via the olfactory pathway).

Passive immunotherapy can involve administration of the antibodies alone or in combination with other chemotherapeutic agents known to treat the disease. For example, passive immunotherapy can be combined with nicotinamide therapy, or with other compounds currently used in the management of Alzheimer's disease, for instance.

In another preferred embodiment, a diseased mammal is vaccinated with diphtheria toxoid, either as a primary series of vaccinations (for those not previously immunized with DT), or as a booster vaccine. Typically, children in the US are given a series of four DPT vaccinations between the ages of 2 months and 6 years. However, the immunity does not last, and booster vaccinations are recommended every 5 to 10 years into adulthood. For the method of the present invention, it is preferred that a booster vaccine is given initially, and the patient monitored periodically for immune status. Preferably, a booster vaccine of diphtheria toxoid is administered intranasally to a human with Alzheimer's disease. The intranasal administration facilitates the initial localization of the immune response to the olfactory neural pathway and the brain. Additionally, the vaccine may contain an adjuvant to enhance the immune response, particularly since immunity is presumed to be somewhat deficient in these neurodegenerative diseases.

The type of adjuvant to be used in the present invention is not critical, as long it is able to increase an antigen-specific immune response of the toxoid(s). Such adjuvants are generally known in the art, and include oil-emulsions, Freunds Complete and Incomplete adjuvant, Vitamin E, aluminum salts or gels, such as aluminum hydroxide, -oxide or -phosphate, saponins, polymers based on polyacrylic acid, such as carbopols, non-ionic block polymers, fatty acid amines, such as avridin and DDA, polymers based on dextran, such as dextran sulphate and DEAE dextran, muramyldipeptides, ISCOMs (immune stimulating complexes, cf. for instance European Patent EP 109942), biodegradable microcapsules, liposomes, bacterial immune stimulators, such as MDP and LPS, glucans and the like (see Altman and Dixon, Advances in Veterinary Science and Comparative Medicine, Vol. 33, 301-343, 1989).

Alum is preferred for human use, although a mucosal adjuvant is preferred for nasal administration. Adjuvants specially suited for mucosal applications are, for example, the E. coli heat-labile toxin (LT) or cholera toxin (CT).

In another aspect of the present invention, described are methods to determine the effectiveness of a particular therapeutic. In essence, these methods test whether the therapeutic agent prevents or reduces ADP-ribosylation by DT or ETA. More particularly, the present invention includes a method for determining if an agent is useful for treating a neurodegenerative disease, comprising conducting an in vitro translation assay, to which has been added diphtheria toxin and the agent to be tested; and determining whether ADP-ribosylation has occurred on the EF-2. Whether the EF-2 is inactivated by the diphtheria toxin, and not prevented by the agent, is determined by examining whether translation has taken place or not, and to what extent (i.e., it can be a quantitative assay). If ADP-ribosylation has not occurred, i.e. if translation product is obtained, then the agent is considered effective as a treatment for neurodegenerative disease according to the present invention.

Several such in vitro translation systems are well known in the art, and even commercially available (for instance, Promega Corp., Madison, Wis.), and the present invention is only limited to the extent that the system is required to contain the mature form of EF2 (i.e., which contains the diphthamide residue).

The translation system should be a eucaryotic system, because the EF-2 is eucaryotic. Preferably, a reticulocyte lysate system, or a lystate of some other cell type such as fibroblasts, is used. To the assay would be added a DNA or RNA molecule that codes for a specific protein, which in the end will be measured to determine the activity of EF-2. In addition, diphtheria toxin or ETA would be added, as well as the agent to be tested. After incubating the reaction mixture for a sufficient time (for example, 8-10 hours), the presence or amount of the specific protein is determined. The determination may be made by way of immunoassay, gel electrophoresis and staining, autoradiography, fluorography, etc., depending on the nature of the protein. For instance, in an especially preferred embodiment for high throughput drug screening, the DNA or RNA added to the system codes for an enzyme, such as luciferase, which can be easily detected with a reagent. Most preferred is the use of an mRNA that codes for green fluorescent protein, whereby translation of the protein is indicated by fluorescence in the tube or well, which can be read by a plate reader, for instance, to get a quantitative measurement, without the need for a reagent. With such an assay system, large numbers of agents can be tested in a short time (i.e., high throughput).

By the above assay, if translation of protein is not significantly inhibited, then the agent is effective in neutralizing the toxin and presumably useful in the treatment of a neurodegenerative disorder, such as Alzheimer's disease. By “not significantly inhibited” is meant that, by comparison to a control assay with toxin only, the agent reduces by 50% the inhibition caused by the toxin. Preferably, the agent reduces the inhibition by 75%, and most preferably by 90%.

The agent to be tested can be any molecule, such as an organic or inorganic compound, or peptidyl or peptide mimetic compound.

As an additional assay, or as a confirmatory assay of agents tested in the in vitro translation assay, is one performed in a cell culture system. For example, on could test whether an agent is useful for treating a neurodegenerative disease by adding to a cell culture that has been transfected with a vector expressing a marker protein an amount of diphtheria toxin which will inhibit translation, and the agent to be tested, and determine if the inhibition of translation by the toxin is blocked by the agent by measuring the marker protein produced. By this method, if translation is not significantly inhibited, then the agent is effective as a treatment for neurodegenerative disease. Preferably, the cell culture contains neuroblastoma cells. Preferably, the marker protein is an enzyme, such as luciferase. Most preferably, the marker protein is green fluorescent protein, which does not require a reagent to measure its presence.

This invention is illustrated in the Examples that follow. These examples are set forth to aid in understanding of the invention but are not intended to, and should not be construed to, limit in any way the invention as set forth in the appended claims.

### EXAMPLES

### Example 1

### Protocol for Clinical Studies

The following is a proposed study to test the clinical effectiveness of the method of treatment for Alzheimer's disease by vaccination with diphtheria vaccine.

Seventy-five patients with early to mid-stage Alzheimer's disease and 75 age-matched normal controls are used as the treatment subjects for the study. Twenty-five from each group would be immunized with a commercially available diphtheria toxin (uncombined) vaccine by intradermal injection. Another twenty-five from each group would be intradermally injected with a phosphate-buffered saline solution. The final 25 from each group would be immunized with a commercially available pneumococcus pneumonia vaccine, an unrelated vaccine which is used as a control in order to ensure that the results seen are in fact due to the diphtheria toxin vaccination and not just a general immune reaction. Pneumococcus vaccine is chosen here because it is a bacteria, but unrelated to diphtheria. In fact, it is a vaccine that is beneficial to people in this age group, because the elderly are susceptible to pneumonia caused by this bacteria and frequently die from complications thereof. Therefore, this vaccine would not be objectionable to the treatment subjects. Both the pneumococcus and diphtheria vaccines are available from Chiron Therapeutics and Vaccines, CA, for instance. If desired, part of the protocol can include 50 late-stage Alzheimer's patients, 25 of whom would receive diphtheria vaccine and 25 only PBS.

Blood samples are taken from all of the subjects at 0, 3, 6 and 12 months, and tested for total serum IgG, and for antibody level to diphtheria toxin. Additionally, at the same time points, each subject is given the Min-Mental State Examination (MMSE), which is the most commonly used test for cognitive function. See Folstein et al., J. Psychiatric Res. 12:189-198 (1975), which is incorporated herein by reference.

At the end of the 12 month period, the MMSE results are analyzed to determine the cognitive abilities of the group of Alzheimer's patients receiving the diphtheria vaccine. It is expected that in this group the cognitive abilities will be recovered or at least remain the same over the course of the 12 month period, and that no significant changes would occur in the control groups. In addition, it is expected that the patient group will, on average, have a significantly lower diphtheria antibody level as compared to the normal controls at time 0 of the study, which will increase at 3, 6 and 12 months. These results would indicate that diphtheria toxin vaccination will effectively treat Alzheimer's disease.

Optionally, the subjects can be tested again at 24 months to determine if the effects are lasting. It is expected that the positive effects will last at least that long, because the diphtheria vaccine normally lasts for 5-10 years.

TABLE 1
Alzheimer's Vaccine Study

| Subjects | Number | Treatment |
|---|---|---|
| Normal Age-Matched | 25 | Diphtheria Toxin Vaccine |
| Controls | 25 | PBS |
| | 25 | Pneumococcus Vaccine |
| Alzheimer Disease Early | 25 | Diphtheria Toxin Vaccine |
| to Mid-Stage | 25 | PBS |
| | 25 | Pneumococcus Vaccine |
| (Optional) Alzheimer | 25 | Diphtheria Toxin Vaccine |
| Disease Late-Stage | 25 | PBS |

### Example 2

In vitro high throughput assay for the screening of drugs that can inhibit ADP-ribosylation of EF-2 by diptheria toxin.

An in vitro translation assay (rabbit reticulocyte lysate system, nuclease treated, available from Promega, Madison, Wis.) is performed using mRNA coding for green-fluorescent protein, or GFP, (produced in a commercially available in vitro transcription assay, also available from Promega) as substrate in a 96/384/1536 well plate format. If necessary, mature form of EF2 is added to the system. The amount of GFP produced is determined by monitoring the fluorescence of GFP. Varying concentrations of diphtheria toxin are added to the assay to determine the amount required to maximally inhibit GFP production. A library of compounds is screened by adding the compounds (approx. 10-100 μM) to reactions with the above determined amount of toxin and determining the amount of GFP produced. Compounds that block the inhibition of GFP translation by the toxin by at least 50% are scored as positive candidate drugs.

For the secondary screening of drug candidates, a cell based assay is used. Neuroblastoma cells (BE-M17) are transfected with a plasmid that codes for the expression of GFP and grown in a 96/384/1536 well plate. Cells are treated with varying amounts DT to determine the amount that maximally inhibits the production of GFP, which is measured by monitoring GFP fluorescence. Varying concentrations of candidate drugs are added to the cultures in the presence of the above determined amount of toxin and GFP production will be monitored. Candidate drugs that block the DT inhibition of GFP production by at least 50% will be scored as positive.

We claim:

1. A method for treating a neurodegenerative disease in a mammal, comprising administering to said mammal an agent which will inhibit and/or reverse ADP-ribosylation of elongation factor-2 (EF-2) in neuronal cells thereof, whereby neuron degeneration is ameliorated.

2. The method of claim 1, wherein said agent comprises nicotinamide or an analog of nicotinamide.

3. The method of claim 1, wherein the agent is administered by aerosol to the nasal passages, orally, or by injection.

4. The method of claim 1, wherein the mammal is a human.

5. The method of claim 1, wherein said agent is a vaccine composition containing diphtheria toxoid.

6. The method of claim 5, wherein the vaccine is administered by spray or aerosol to the nasal passages.

7. The method of claim 5, wherein the dosage of vaccine is comparable to a typical booster vaccine.

8. The method of claim 5, wherein the vaccine contain an adjuvant.

9. The method of claim 1, wherein the agent is antitoxin to C. diphtheria exotoxin, which is administered as passive immunotherapy.

10. The method of claim 9, wherein the antitoxin is purified antibody to diphtheria toxin.

11. The method of claim 9, wherein the antitoxin is a monoclonal antibody.

12. The method of claim 11, wherein the antibody is a variable region fragment thereof.

13. The method of claim 11, wherein the antitoxin is an antibody which has an increased affinity for ETA or DT as compared to its wildtype counterpart.

14. The method of claim 13, wherein the antibody is a single chain Fv antibody.

15. A method for determining if an agent is useful for treating a neurodegenerative disease, comprising:
(a) performing a eucaryotic in vitro translation assay, to which has been added nucleic acid that codes for a marker protein, diphtheria toxin or exotoxin A, and the agent to be tested; and
(b) determining if the inhibition of translation by the toxin is blocked by the agent by measuring the marker protein produced;
wherein if translation is not blocked, then the agent is effective as a treatment for neurodegenerative disease.

16. The method of claim 15, wherein the neurodegenerative disease is Alzheimer's Disease.

17. The method of claim 15, wherein the marker protein is an enzyme.

18. The method of claim 17, wherein the enzyme is luciferase.

19. The method of claim 15, wherein the marker protein is green fluorescent protein.

20. An agent effective in preventing inhibition of translation by diphtheria toxin or exotoxin A as determined by the method of claim 15.

21. A method for determining if an agent is useful for treating a neurodegenerative disease, comprising:
(a) adding to a cell culture that has been transfected with a vector that expresses a marker protein an amount of diphtheria toxin which will inhibit translation, and the agent to be tested;
(b) determining if the inhibition of translation by the toxin is blocked by the agent by measuring the marker protein produced;
wherein if translation is not blocked, then the agent is effective as a treatment for neurodegenerative disease.

22. The method of claim 21, wherein the neurodegenerative disease is Alzheimer's Disease.

23. The method of claim 21, wherein the cell culture contains neuroblastoma cells.

24. The method of claim 21, wherein the marker protein is an enzyme.

25. The method of claim 24, wherein the enzyme is luciferase.

26. The method of claim 21, wherein the marker protein is green fluorescent protein.
